(19) United States
(12) Patent Application Publication
(10) Pub. No.: US 2010/0311983 A1
(43) Pub. Date: Dec. 9, 2010

(54) PROCESS FOR THE PREPARATION OF 2-CYANOIMINO-1, 3-THIAZOLIDINE

(75) Inventors: Nagarajan Kuppuswamy, Bangalore (IN); Rajender Pershad Gupta, Bangalore (IN); Bhupathiraju Naga Satyanarayanaraju, Bangalore (IN); Somu Madhava Reddy, Bangalore (IN); Seranthimata Samshuddin, Bangalore (IN); K. P. Jadhav, Bangalore (IN); Sham Dhamnaskar, Bangalore (IN)

Correspondence Address: CHOATE, HALL & STEWART LLP, TWO INTERNATIONAL PLACE, BOSTON, MA, 02110, US

(73) Assignee: HIKAL LIMITED, Bangalore (IN)

(21) Appl. No.: 12/865,796

(22) PCT Filed: Feb. 2, 2009
(86) PCT No.: PCT/IN09/00070

§ 371 Date: Aug. 2, 2010

(30) Foreign Application Priority Data
Feb. 1, 2008 (IN) 267/CHE/2008
Feb. 1, 2008 (IN) 268/CHE/2008
Feb. 1, 2008 (IN) 269/CHE/2008

(51) Int. Cl. C07D 277/18

(52) U.S. Cl. 548/198

(57) ABSTRACT

The present invention relates to a process for the preparation of substantially pure 2-cyanoimino-1,3-thiazolidine of Formula (I) by cyclization of dimethyl N-cyanoiminodithiocarbonate with 2-aminoethanethiol or the salt thereof in the presence of an alkali metal alkoxide. Further the present invention provides a process for the preparation of substantially pure 2-cyanoimino-1,3-thiazolidine of Formula (I) by cyclization of dimethyl N-cyanoiminodithiocarbonate with 2-aminoethanethiol or the salt thereof in the presence of aqueous ammonia.

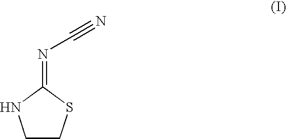

### FIELD OF THE INVENTION

The present invention provides a process for the preparation of substantially pure 2-cyanoimino-1,3-thiazolidine of Formula-I,

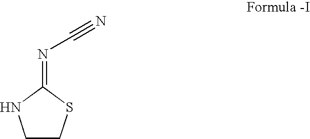

by cyclisation of dimethyl N-cyanoiminodithiocarbonate ester and 2-aminoethanethiol or salts thereof in presence of base solution.

Further the present invention also provides a process for the preparation of substantially pure 2-cyanoimino-1,3-thiazolidine of Formula-I, by cyclisation of dimethyl N-cyanoiminodithiocarbonate ester and 2-aminoethanethiol or salts thereof in presence of aqueous ammonia.

### BACKGROUND AND PRIOR ART

The methods for preparation of 2-cyanoimino-1,3-thiazolidine from the reaction between dimethyl N-cyanoiminodithiocarbonate ester and 2-aminoethanethiol, are those described in Arch. pharm. (weiheim, Ger.), 305(10), P731 (1972), Japanese unexamined patent publication (kokai) No. 48-91064, Gazz.Chim.Ital., 110 (5-6), P345, and WO92-17462 (1992). This reaction is considered to follow the following scheme:

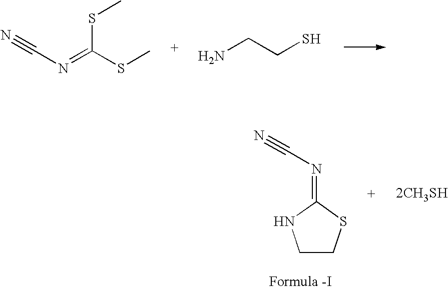

J. Heterocycl. Chem., 24(1), P275 (1987) describes a method for preparing 2-cyanoimino-1,3-thiazolidine from the reaction between diphenyl N-cyanoiminodithiocarbonate and 2-aminoethanethiol. The drawback of this process is using the relatively expensive diphenyl N-cyanoiminodithiocarbonate compound. This reaction is depicted in the following scheme.

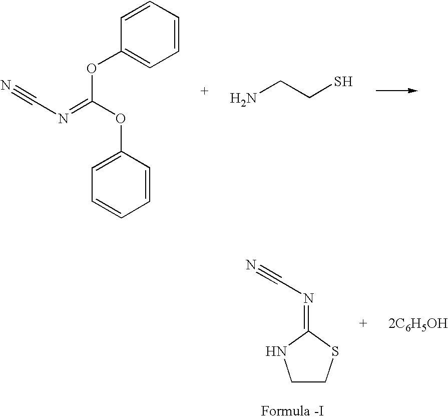

Org. Prep. Procedure Int. 23, (6), 721-728 (1991) describes a method for preparing cyanoimino-1,3-thiazolidine in 48% yield which comprises stirring dimethyl N-cyanoimidocarbonate for a prolonged period at a pH of 10-11 with cysteamine in aqueous sodium hydroxide solution. The melting point of the product thus obtained (m.p. 168°-170° C.), however, differs considerably from that of pure 2-cyanoimino-1,3-thiazolidine (m.p. 154°-156° C.), since the former is probably contaminated by secondary products. Therefore, further purification would reduce still further the yield from the reported 48%, so that this process is unsuitable for industrial production.

U.S. Pat. No. 5,574,165 discloses a two-step process for preparing 2-cyanoimino-1,3-thiazolidine, said process comprising; reacting 2-aminoethanethiol or salt thereof and dialkyl N-cyanoimidocarbonate in a diluent, in the presence of a base such as sodium hydroxide, potassium hydroxide, calcium hydroxide, sodium carbonate, potassium carbonate, sodium methylate, potassium methylate, sodium ethylate or potassium ethylate, in the presence of a protective-gas atmosphere to obtain an intermediate which is subsequently cyclized at a pH of from 8 to 9.5 to obtain 2-cyanoimino-1,3-thiazolidine. In this process the purity of the product is only 95.8%. This reaction is depicted in the following scheme:

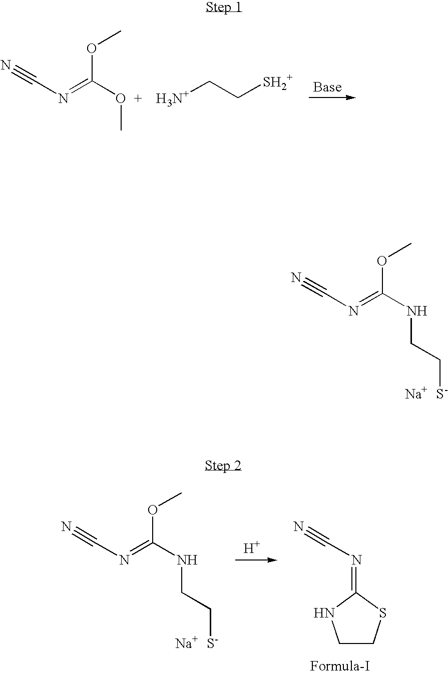

U.S. Pat. No. 5,591,859 discloses a process for preparation of 2-cyanoimino-1,3-thiazolidine, said process comprising; reacting 2-aminoethanethiol or salt thereof (wherein 2-aminoethanethiol is released by addition of a base) and dialkyl N-cyanoimidocarbonate in water and/or an organic solvent in a pH range from 7 to 12, in the presence of a protective-gas atmosphere and completing the cyclization at a pH of >8 by addition of a base such as alkali hydroxides, alkali carbonates and strongly basic amines in aqueous solution such as triethylamine and diethylamine to obtain 2-cyanoimino-1,3-thiazolidine.

U.S. Pat. No. 6,858,737 discloses a process for the preparation of 2-cyanoimino-1,3-thiazolidine comprising the cyclization reaction of dimethyl N-cyanoiminodithiocarbonate with 2-aminoethanethiol or the salt thereof in the presence of an alkali metal hydroxide. This process is limited to the scope of using alkali metal hydroxide as a base and water as the solvent.

### SUMMARY OF THE INVENTION

The present invention provides a process for the preparation of substantially pure 2-cyanoimino-1,3-thiazolidine of Formula-I,

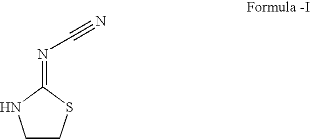

said process comprises, dissolving a base in solvent; adding 2-aminoethanethiol or salt thereof of Formula-II

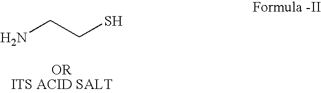

and dimethyl N-cyanoiminodithiocarbonate ester of Formula-III

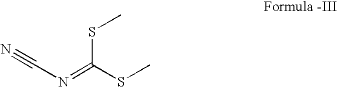

to said solution of base in inert atmosphere to obtain a reaction mixture; and adjusting pH of said reaction mixture with a mineral acid such as hydrochloric acid, followed by heating to obtain substantially pure 2-cyanoimino-1,3-thiazolidine of Formula-I.

Further the present disclosure provides a process for the preparation of 2-cyanoimino-1,3-thiazolidine of Formula-I, said process comprises:
a. Dissolving dimethyl N-cyanoiminodithiocarbonate ester of Formula-II in a solvent to obtain a solution of dimethyl N-cyanoiminodithiocarbonate ester of Formula-II;
b. Adding 2-aminoethanethiol or salt thereof of Formula-III and aqueous ammonia to said solution of dimethyl N-cyanoiminodithiocarbonate ester of Formula-II
c. Performing the above reaction at 5° C. to ambient temperature for 2 hours to obtain substantially pure 2-cyanoimino-1,3-thiazolidine of Formula-I.

These and other features, aspects, and advantages of the present subject matter will become better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### DETAILED DESCRIPTION OF THE INVENTION

Accordingly, the present invention provides a process for the preparation of substantially pure 2-cyanoimino-1,3-thiazolidine of Formula-I,

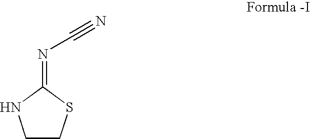

said process comprising:
1. dissolving a base in a solvent to obtain a solution of the base;
2. adding 2-aminoethanethiol or salt thereof of Formula-II

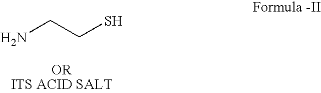

and dimethyl N-cyanoiminodithiocarbonate ester of Formula-III

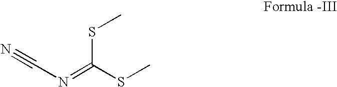

to said solution of the base in an inert atmosphere to obtain a reaction mixture; and
3. adjusting pH of said reaction mixture with a mineral acid, stirring followed by heating to obtain substantially pure 2-cyanoimino-1,3-thiazolidine of Formula-I wherein the molar ratio of base to 2-aminoethanethiol or salts thereof is from 1:1 to 1:1.5 and the molar ratio of dimethyl N cyanoiminothiocarbonate to 2-aminoethanethiol or salts thereof is from 1:0.95 to 1:1.15.

The solvent used in the process of the present disclosure is selected from C1-C4 straight or branched chain alcohol, tetrahydrofuran, toluene, water or a mixture thereof.

The mineral acid used in the present disclosure is selected from hydrochloric acid, sulfuric acid, nitric acid, carbonic acid or phosphoric acid.

In an embodiment of the present disclosure, the base used is an alkali metal alkoxide or an inorganic carbonate.

In an embodiment, the present disclosure provides a process for the preparation of substantially pure 2-cyanoimino-1,3-thiazolidine of Formula-I, said process comprising:
a. dissolving alkali metal alkoxide in an organic solvent to obtain a solution of alkali metal alkoxide;
b. adding 2-aminoethanethiol or salt thereof of Formula-II and dimethyl N-cyanoiminodithiocarbonate ester of Formula-III; to said solution of alkali metal alkoxide in inert atmosphere to obtain a reaction mixture; and
c. adjusting pH of said reaction mixture with a mineral acid followed by heating to obtain substantially pure 2-cyanoimino-1,3-thiazolidine of Formula-I.

The reaction of the present invention is depicted in the form of the following scheme

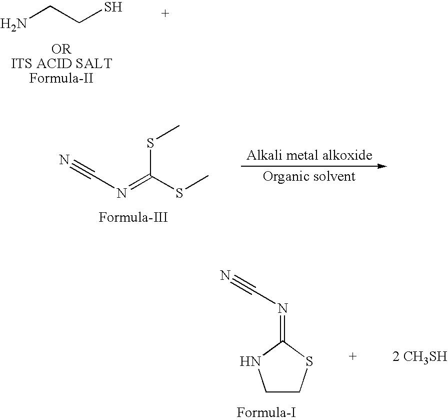

At the time of reaction, the dimethyl N-cyanoiminodithiocarbonate is gradually added so that the temperature of the reaction mixture does not rise above 5° C. After the completion of the addition of dimethyl N-cyanoiminothiocarbonate ester, the cyclisation reaction is preferably performed at 0° C. to 5° C.

The reaction ratio of dimethyl N-cyanoiminothiocarbonate and 2-aminoethanethiol, by molar ratio is, preferably 1:0.95 to 1.15, more preferably 1:0.99 to 1.1. If the amount of the 2-aminoethanethiol is too large, an undesirable polymerization reaction occurs resulting in lower yield.

The reaction time of the cyclisation reaction is not particularly limited, but for example is 10 minutes to 5 hours, preferably 1 to 3 hours. If the cyclisation reaction time is too short, the cyclisation reaction does not proceed sufficiently, while if too long, the reaction will not proceed further, and therefore this is not wise economically.

After the end of the reaction, the temperature of the reaction mixture is raised to 10° to 30° C., preferably to about 20° C. then the system is adjusted to a pH of 3 to 10 by a suitable acid, preferably 3 to 6. After the adjustment of the pH, an operation is performed to gradually raise the temperature in the system to about 40° C. and remove the byproduct that is methyl mercaptan, from the system. It is to be noted that, when removing the methyl mercaptan from the system, the rapid temperature rise becomes a cause of violent bubbling etc., and therefore, this is not preferred. Further, the temperature is not particularly limited, but after the operation for removing the methyl mercaptan from the system is over, the system is cooled to approximately room temperature, the operation of this procedure at a high temperature is not preferable in view of the process time. A temperature of about 35° C. to about 45° C. is preferable.

Also, the above-mentioned stirring time is not particularly limited, but the treatment is preferably completed for preferably 1 to 5 hours, more preferably 2 to 3 hours. By removing the byproduct methyl mercaptan from the system in vacuo during the stirring, it is possible to reduce the odor of the product and the surrounding environment.

According to the present disclosure, the resultant reaction mixture containing the 2-cyanoimino-1,3-thiazolidine obtained is again cooled to about 0° C. to about 5° C., preferably about 0° C., then the resultant 2-cyanoimino-1,3-thiazolidine is filtered and then washed. This washing can be performed with water or another solvent (for example, methanol, ethanol, etc.), but from the viewpoints of economy and environment, the washing with water is preferred.

The drying conditions of the 2-cyanoimino-1,3-thiazolidine thus produced are also not particularly limited and also differ depending on the type of the dryer, but when using a box type dryer, the drying is preferably carried out at a drying temperature of 60 to 120° C., more preferably 80 to 100° C. for a drying time of preferably 30 minutes to 24 hours, more preferably 3 to 6 hours, and a degree of vacuum of preferably 25 mmHg or less.

As specific examples of mineral acids used for controlling the pH of the reaction mixture, hydrochloric acid, sulfuric acid, nitric acid, carbonic acid, phosphoric acid, etc. may be mentioned, but from an economical viewpoint, the use of hydrochloric acid or sulfuric acid is preferred.

In an embodiment of the present disclosure, the ratio of dimethyl N-cyanoiminothiocarbonate and acid salt of 2-aminoethanethiol by molar ratio is preferably 1:0.95 to 1.15.

An embodiment of the present disclosure, base used is alkali metal alkoxide and is first dissolved in an organic solvent. Specific examples of the alkali metal alkoxides used in the present invention are sodium methoxide, sodium ethoxide, potassium methoxide, potassium ethoxide, sodium butoxide, sodium isobutoxide, potassium butoxide, or the like, preferably sodium methoxide is used. These alkali metal alkoxides are used in amounts of preferably 1 to 1.5 moles based upon 1 mole of acid salt of 2-aminoethanethiol.

Another embodiment of the present disclosure the organic solvent is a lower straight or branched chain alcohol consisting of C1-C4 carbon atoms, tetrahydrofuran or toluene or a mixture thereof. The lower straight or branched chain alcohol consisting of C1-C4 carbon atoms is methanol, ethanol, propanol, isopropanol, and the like.

Yet another embodiment of the present disclosure the acid salt of 2-aminoethanethiol is selected from the group of hydrochloride, sulfate, nitrate, carbonate, acetate or the like. Any salt alone or any combination thereof may be used, but the use of a mineral acid salt is preferable from the viewpoint of the reactivity, while a hydrochloride is more preferable from the viewpoint of solubility, and economy. The acid salt of 2-aminoethanethiol is added to an alkali metal alkoxide preferably in such an amount that the molar ratio with the alkali metal alkoxide comes in the above range, and then the mixture is cooled to 0 to 5° C., preferably to 0° C.

In an embodiment of the present disclosure the molar ratio of alkali metal alkoxide to acid salt of 2 aminoethanethiol is from 1:1 to 1.5:1

In another embodiment of the present disclosure provides a process for the preparation of substantially pure 2-cyanoimino-1,3-thiazolidine of Formula-I, said process comprising:
a. dissolving an inorganic carbonate in solvent to obtain a solution of inorganic carbonate;
b. adding 2-aminoethanethiol or salt thereof of Formula-II and dimethyl N-cyanoiminodithiocarbonate ester of Formula-III to said solution of inorganic carbonate to obtain a reaction mixture; and
c. Adjusting pH of said reaction mixture with a mineral acid followed by heating, to obtain substantially pure 2-cyanoimino-1,3-thiazolidine of Formula-I wherein the molar ratio of inorganic carbonate to 2 aminoethanethiol salt is from 1:1.1 to 1.5:1.1 and the molar ratio of dimethyl N-cyanoiminodithiocarbonate ester to 2-aminoethanethiol acid salt is from 1:0.95 to 1:1.15.
   The reaction of the present invention is depicted in the form of the following scheme;

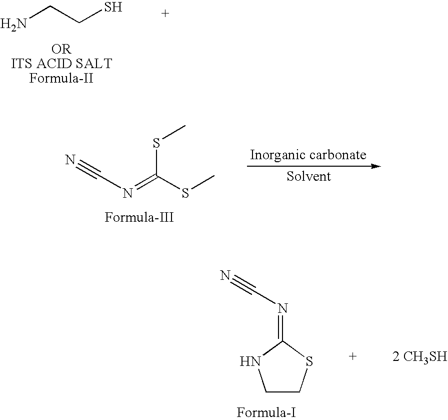

An embodiment of the present disclosure, the base used is an inorganic carbonate and first dissolved in a solvent. Specific examples of the inorganic carbonate used in the present disclosure are alkali or alkaline earth metal carbonates selected from sodium carbonate, potassium carbonate, magnesium carbonate, calcium carbonate and ammonium carbonate and the like. The inorganic carbonates are used in amounts of preferably 1 to 1.5 moles based upon 1.1 mole of acid salt of 2-aminoethanethiol.

According to the present disclosure, at the time of reaction the dimethyl N-cyanoiminodithiocarbonate is gradually added so that the temperature of the reaction mixture does not rise above 5° C. After the completion of the addition of the dimethyl N-cyanoiminothiocarbonate ester, the cyclisation reaction is preferably performed at 0° C. to 5° C.

The reaction ratio of the dimethyl N-cyanoiminothiocarbonate and acid salt of 2-aminoethanethiol, by molar ratio is, preferably 1:0.95 to 1.15, more preferably 1:0.99 to 1.1. If the amount of the 2-aminoethanethiol is too large, an undesirable polymerization reaction occurs resulting in lower yield.

The reaction time of the cyclisation reaction is not particularly limited, but for example is 10 minutes to 5 hours, preferably 1 to 3 hours. If the cyclisation reaction time is too short, the cyclisation reaction does not proceed sufficiently, while if too long, the reaction will not proceed further, and therefore this is not wise economically.

After the end of the reaction, the temperature of the reaction mixture is raised to 10 to 30° C., preferably to about 20° C. then the system is adjusted to a pH of 3 to 10 by a suitable acid, preferably 3 to 6. After the adjustment of the pH, an operation is performed to gradually raise the temperature in the system to about 40° C. and remove the byproduct, methyl mercaptan, from the system. It is to be noted that, when removing the methyl mercaptan from the system, the rapid temperature rise becomes a cause of violent bubbling etc., and therefore, this is not preferred. Further, the temperature is not particularly limited, but after the operation for removing the methyl mercaptan from the system is over, the system is cooled to approximately room temperature, the operation of this procedure at a high temperature is not preferable in view of the process time. A temperature of about 35° C. to about 45° C. is preferable.

Also, the above-mentioned stirring time is not particularly limited, but the treatment is preferably completed for preferably 1 to 5 hours, more preferably 2 to 3 hours. By removing the byproduct methyl mercaptan from the system in vacuo during the stirring, it is possible to reduce the odor of the product and the surrounding environment.

According to the present disclosure, the resultant reaction mixture containing the 2-cyanoimino-1,3-thiazolidine obtained is again cooled to about 0° C. to about 5° C., preferably about 0° C., then the resultant 2-cyanoimino-1,3-thiazolidine is filtered and then washed. This washing can be performed with water or another solvent (for example, methanol, ethanol, etc.), but from the viewpoints of economy and environment, the washing with water is preferred.

The drying conditions of the 2-cyanoimino-1,3-thiazolidine thus produced are also not particularly limited and also differ depending on the type of the dryer, but when using a box type dryer, the drying is preferably carried out at a drying temperature of 60 to 120° C., more preferably 80 to 100° C. for a drying time of preferably 30 minutes to 24 hours, more preferably 3 to 6 hours, and a degree of vacuum of preferably 25 mmHg or less.

As specific examples of the mineral acids used for controlling the pH of the reaction mixture, hydrochloric acid, sulfuric acid, nitric acid, carbonic acid, phosphoric acid, etc. may be mentioned, but from an economical viewpoint, the use of hydrochloric acid or sulfuric acid is preferred.

Another embodiment of the present disclosure, the solvent used is a lower straight or branched chain alcohol consisting of C₁-C₄ carbon atoms, water or mixture thereof. The lower straight or branched chain alcohol consisting of C₁-C₄ carbon atoms is methanol, ethanol, propanol, isopropanol, and the like.

Yet another embodiment of the present disclosure the acid salt of 2-aminoethanethiol is hydrochloride, sulfate, nitrate, carbonate, acetate or the like. Any salt alone or any combination thereof may be used, but the use of a mineral acid salt is preferable from the viewpoint of the reactivity, while a hydrochloride is more preferable from the viewpoint of solubility, and economy. The acid salt of 2-aminoethanethiol is added to an inorganic carbonate preferably in such an amount that the molar ratio with the inorganic carbonate comes in the above range, and then the mixture is cooled to 0 to 5° C., preferably to 0° C.

Further, the present disclosure provides a process for the preparation of substantially pure 2-cyanoimino-1,3-thiazolidine of Formula-I, said process comprising:
a. Dissolving dimethyl N-cyanoiminodithiocarbonate ester of formula H in a solvent, to obtain a solution of dimethyl N-cyanoiminodithiocarbonate ester of Formula-II;
b. Adding 2-aminoethanethiol or salts thereof of formula III to a mixture of water and aqueous ammonia
c. reacting the solution of ester of formula II and solution thiol of formula III at 0-35° C. for 2 hours to obtain a precipitate which is then recrystallised from hot water to obtain substantially pure of 2-cyanoimino-1,3-thiazolidine, wherein the molar ratio of dimethyl N-cyanoiminodithiocarbonate ester to 2-aminoethanethiol acid salt is from 1:0.95 to 1:1.15.

The reaction of the present invention is depicted in the form of the following scheme

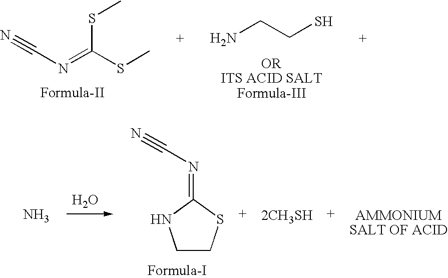

In the present disclosure dimethyl N-cyanoiminodithiocarbonate of Formula-II is first dissolved in a solvent such as water and the reaction mixture is stirred at 0° to 10° C. Then acid salt of 2-aminoethanethiol dissolved in water is added to the reaction mixture in one lot followed by the addition of aqueous ammonia solution. The cyclisation reaction commences at 5° C., which is evident from the evolution of the equimolar quantity of methylmercaptan gas and slight exotherm during the reaction.

In an embodiment of the present disclosure performing the above reaction at 5° C. to ambient temperature for 2 hours to obtain substantially pure 2-cyanoimino-1,3-thiazolidine of Formula-I.

In another embodiment of the present disclosure, the aqueous ammonia is 18-25% w/w.

In an embodiment of the present disclosure the solvent is water.

In yet another embodiment of the present disclosure the quantity of aqueous ammonia and water in the said mixture is equal.

It is an embodiment of the present disclosure the reaction ratio of the dimethyl N-cyanoiminodithiocarbonate and acid salt of 2-aminoethanethiol, by molar ratio is, preferably 1:1. If the amount of the 2-aminoethanethiol is too large, an undesirable polymerization reaction occurs resulting in lower yield.

Another embodiment of the present disclosure the methyl mercaptan gas can be trapped in sodium methoxide solution or 10% caustic lye solution to avoid obnoxious, penetrating odour of methyl mercaptan and isolated as sodium salt as a valuable by product for other agrochemicals.

Yet another embodiment of the present disclosure the acid salt of 2-aminoethanethiol is hydrochloride, which is most preferable from the viewpoint of commercial availability.

The time of the cyclisation reaction is not particularly limited, but for example is 10 mins. to 5 hours, preferably 1 to 3 hours. If the cyclisation reaction time is too short, the cyclisation minutes reaction does not proceed sufficiently, while if too long, the reaction will not proceed further, and therefore this is not wise economically.
The process as claimed in claim 8 in which 2-cyanoimino-1,3-thiazolidine is subjected to recrystallised from hot water to obtain substantially pure form of 2-cyanoimino-1,3-thiazolidine.

In an embodiment of the present disclosure time of reaction is 10 min to 5 hrs.

In another embodiment of the present disclosure the pH of the reaction mixture is 3-10.

Although the subject matter has been described in considerable detail with reference to certain preferred embodiments thereof, other embodiments are possible. As such, the spirit and scope of the appended claims should not be limited to the description of the preferred embodiment contained therein.

### Example

The disclosure will now be illustrated with working examples, which is intended to illustrate the working of disclosure and not intended to take restrictively to imply any limitations on the scope of the present disclosure.

### Example 1

A 100 ml four-necked flask provided with a thermometer and a stirrer is charged with 8 g of methanol (0.25 moles) and 2.5 g of sodium methoxide (0.046 mole, 1.36 moles based upon 1.1 mole of 2-aminoethane thiol hydrochloride) under nitrogen. The mixture is cooled and stirred to dissolve in the methanol. Then, 4.2 g of 2-aminoethane thiol hydrochloride (0.037 moles) was added thereto and dissolved therein, and the reaction mixture is cooled to 0° C. To this reaction mixture, 5 g of dimethyl N-cyanoiminodithiocarbonate (0.034 moles) is added keeping the inside temperature at 5° C. or less. After the end of addition, the mixture is allowed to react at 0 to 5° C. for 2 hours under nitrogen. Thereafter, the reaction mixture is heated to 20° C. and pH is adjusted to 4 with aqueous hydrochloric acid solution (36% w/w), then further heated to 40° C. and stirred for 2 hours. After stirring, the reaction mixture was cooled to 0° C. and the crystals were suction filtered and washed with 15 ml of water to obtain 3.8 g of 2-cyanoimino-1,3-thiazolidine. The wet crystals were dried in vacuo at 80° C. under reduced pressure for 5 hours to obtain 3.5 g of 2-cyanoimino-1,3-thiazolidine (yield 80%) with 99.9% HPLC purity.

### Example 2

A 100 ml four-necked flask provided with a thermometer and a stirrer is charged with 4 g of methanol and 6.5 ml of 30% sodium methoxide solution (0.0342 mole, 1 mole equivalent based upon 1.1 mole equivalent of 2-aminoethane thiol hydrochloride) under nitrogen. The mixture is cooled and stirred to dissolve in methanol, then 4.2 g of 2-aminoethane thiol hydrochloride (0.037 moles) is added thereto and dissolved therein. The reaction mixture is cooled to 0° C. and 5 g of dimethyl N-cyanoiminodithiocarbonate (0.034 moles) is added keeping the inside temperature at 5° C. or less. After the end of addition, the mixture is allowed to react at 0 to 5° C. for 2 hours. Thereafter, the reaction mixture is heated to 20° C. and pH is adjusted to 4 with aqueous hydrochloric acid solution (36% w/w) and further heated to 40° C. and stirred for 2 hours. After stirring, the reaction mixture was cooled to 0° C. and the crystals are suction filtered and washed with 15 ml of water to obtain 3.8 g of 2-cyanoimino-1,3-thiazolidine. The wet crystals are dried in vacuo at 80° C. under reduced pressure for 5 hours to obtain 3.5 g of 2-cyanoimino-1,3-thiazolidine (yield 80%) with >99% HPLC purity.

### Example 3

A 1 litre four-necked flask provided with a thermometer and stirrer is charged with 90 g of methanol (2.815 moles) and 36 g of sodium methoxide (0.667 moles, 1.3 mole equivalent based upon 1.1 mole equivalent of 2-aminoethane thiol hydrochloride) under nitrogen. The mixture is cooled and stirred to dissolve in methanol, then 63.5 g of 2-aminoethane thiol hydrochloride (0.564 moles) is added thereto and dissolved therein. The reaction mixture is cooled to 0° C. and 75 g of dimethyl N-cyanoiminodithiocarbonate (0.514 moles) is added keeping the inside temperature at 5° C. or less. After the end of addition, the mixture is allowed to react at 0 to 5° C. for 2 hours under nitrogen. Thereafter, the reaction mixture is heated to 20° C. and pH is adjusted to 4 with aqueous hydrochloric acid solution (36% w/w), then further heated to 40° C. and stirred for 2 hours. After stirring, the reaction mixture is cooled to 0° C. and the crystals are suction filtered and the slurry thus obtained is washed with 225 ml of chilled water to obtain 66 g of 2-cyanoimino-1,3-thiazolidine. The wet crystals are dried in vacuo at 80° C. under reduced pressure for 5 hours to obtain 58 g of 2-cyanoimino-1,3-thiazolidine (yield 85.8%) with 99.9% HPLC purity.

### Example 4

A 1 litre four-necked flask provided with a thermometer and stirrer is charged with 60 g of ethanol (1.304 moles) and 30.2 g of sodium ethoxide (0.444 moles, 1.3 mole equivalent based upon 1.1 mole equivalent of 2-aminoethane thiol hydrochloride) under nitrogen. The mixture is cooled and stirred to dissolve in ethanol and then 42.3 g of 2-aminoethane thiol hydrochloride (0.376 moles) is added thereto and dissolved therein. The reaction mixture is cooled to 0° C. and 50 g of dimethyl N-cyanoiminodithiocarbonate (0.342 moles) is added keeping the inside temperature at 5° C. or less. After the end of addition, the mixture is allowed to react at 0 to 5° C. for 2 hours under nitrogen. Thereafter, the reaction mixture is heated to 20° C. and pH is adjusted to 4 with aqueous hydrochloric acid solution (36% w/w), then further heated to 40° C. and stirred for 2 hours. After stirring, the reaction mixture is cooled to 0° C. and the crystals are suction filtered and the slurry thus obtained is washed with 150 ml of chilled water to obtain 44 g of 2-cyanoimino-1,3-thiazolidine. The wet crystals are dried in vacuo at 80° C. under reduced pressure for 5 hours to obtain 36 g of 2-cyanoimino-1,3-thiazolidine (yield 83%) with 99.5% HPLC purity.

### Example 5

A 100 ml four-necked flask provided with a thermometer and stirrer is charged with 7 g of toluene (0.076 moles) and 1 g of sodium methoxide (0.0185 moles, 1.36 mole equivalent based upon 1.1 mole equivalent of 2-aminoethane thiol hydrochloride) under nitrogen. The mixture is cooled and stirred to dissolve in toluene, then 1.73 g of 2-aminoethane thiol hydrochloride (0.016 moles) is added thereto and dissolved therein. The reaction mixture is cooled to 0° C. and 2 g of dimethyl N-cyanoiminodithiocarbonate (0.0136 moles) is added keeping the inside temperature at 5° C. or less. After the end of addition, the mixture is allowed to react at 0 to 5° C. for 2 hours under nitrogen. Thereafter, the reaction mixture is heated to 20° C. and pH is adjusted to 4 with aqueous hydrochloric acid solution (36% w/w), then further heated to 40° C. and stirred for 2 hours. After stirring, the reaction mixture is cooled to 0° C. and the crystals are suction filtered and the slurry thus obtained is washed with 6 ml of chilled water to obtain 1.1 g of 2-cyanoimino-1,3-thiazolidine. The wet crystals are dried in vacuo at 80° C. under reduced pressure for 5 hours to obtain 0.9 g of 2-cyanoimino-1,3-thiazolidine (yield 52%) with >99% HPLC purity.

### Example 6

A 100 ml four-necked flask provided with a thermometer and stirrer is charged with 6.2 g of tetrahydrofuran (0.0861 moles) and 1 g of sodium methoxide (0.0185 moles, 1.36 mole equivalent based upon 1.1 mole equivalent of 2-aminoethane thiol hydrochloride) under nitrogen. The mixture is cooled and stirred to dissolve in the tetrahydrofuran (THF) and then 1.73 g of 2-aminoethane thiol hydrochloride (0.016 moles) is added thereto and dissolved therein. The reaction mixture is cooled to 0° C. and 2 g of dimethyl N-cyanoiminodithiocarbonate (0.0136 moles) is added keeping the inside temperature at 5° C. or less. After the end of addition, the mixture is allowed to react at 0 to 5° C. for 2 hours under nitrogen. Thereafter, the reaction mixture is heated to 20° C. and pH is adjusted to 4 with aqueous hydrochloric acid solution (36% w/w), then further heated to 40° C. and stirred for 2 hours. After stirring, the reaction mixture is cooled to 0° C. and the crystals are suction filtered and the slurry thus obtained is washed with 6 ml of chilled water to obtain 1.3 g of 2-cyanoimino-1,3-thiazolidine. The wet crystals were dried in vacuo at 80° C. under reduced pressure for 5 hours to obtain 1.1 g of 2-cyanoimino-1,3-thiazolidine (yield 63%) with >99% HPLC purity.

### Example 7

A 100 ml four-necked flask provided with a thermometer and a stirrer is charged with 8 ml of water, then 1.16 g of sodium carbonate (0.015 mole, 1.1 mole equivalent based upon 1.1 mole equivalent of 2-aminoethane thiol hydrochloride). The mixture is cooled and stirred to dissolve in the water. Then, 1.73 g of 2-aminoethane thiol hydrochloride (0.037 moles) is added thereto and dissolved therein. The reaction mixture is cooled to 0° C. and 2 g of dimethyl N-cyanoiminodithiocarbonate (0.034 moles) is added keeping the temperature inside the system at 5° C. or less. After the end of addition, the mixture is allowed to react at 0 to 5° C. for 2 hours. Thereafter, the reaction mixture is heated to 20° C. and its pH is adjusted to 4 by careful addition of aqueous hydrochloric acid solution (36% w/w). The reaction mass is then further heated to 40° C. and stirred for 2 hours and cooled to 0° C. with stirring. The resultant crystals are suction filtered and the slurry thus obtained is washed with 15 ml of chilled water to obtain 1.6 g of 2-cyanoimino-1,3-thiazolidine. The wet crystals are dried in vacuo at 80° C. under reduced pressure for 5 hours to obtain 1.4 g of 2-cyanoimino-1,3-thiazolidine (yield 80%) with >99% HPLC purity.

### Example 8

A 100 ml four-necked flask provided with a thermometer and a stirrer is charged with 16 g of methanol, and 4 g of sodium carbonate (0.037 moles, 1.1 mole equivalent based upon 1.1 mole equivalent of 2-aminoethane thiol hydrochloride). The mixture is cooled and stirred to be dissolved in the methanol, then 4.2 g of 2-aminoethane thiol hydrochloride (0.037 moles) is added thereto and dissolved therein. The reaction mixture is now cooled to 0° C. To this reaction mixture, 5 g of dimethyl N-cyanoiminodithiocarbonate (0.034 moles) is added keeping the temperature inside the system at 5° C. or less. After the end of addition, the mixture is allowed to react at 0 to 5° C. for 2 hours. Thereafter, the reaction mixture is heated to 20° C., the pH is adjusted to 4 by careful addition of aqueous hydrochloric acid solution (36% w/w), the reaction mass then further heated to 40° C. and stirred for 2 hours. It is cooled to 0° C. under stirring. The precipitated crystals are suction filtered and slurry washing with 15 ml of chilled water to obtain 3.3 g of 2-cyanoimino-1,3-thiazolidine. The wet crystals are dried in vacuo at 80° C. under reduced pressure for 5 hours to obtain 3.1 g of 2-cyanoimino-1,3-thiazolidine (yield 72%) with >99% HPLC purity.

### Example 9

A 100 ml four-necked flask provided with a thermometer and a stirrer is charged with 8 ml of water, and 2.15 g of potassium carbonate (0.0156 moles, 1.1 mole equivalent based upon 1.1 mole equivalent of 2-aminoethane thiol hydrochloride). The mixture is cooled and stirred to dissolve in water, then 1.73 g of 2-aminoethane thiol hydrochloride (0.037 moles) is added thereto and dissolved therein, the reaction mixture is now cooled to 0° C. To this reaction mixture, 2 g of dimethyl N-cyanoiminodithiocarbonate ester (0.034 moles) is added keeping the temperature inside the system at 5° C. or less. After the end of addition, the mixture is allowed to react at 0 to 5° C. for 2 hours. Thereafter, the reaction mixture is heated to 20° C., the pH is adjusted to 4 with aqueous hydrochloric acid solution (36% w/w), the reaction mass then further heated to 40° C. and stirred for 2 hours. It is cooled to 0° C., under stirring the precipitated crystals are suction filtered and slurry washing with 15 ml of chilled water to obtain 1.3 g of 2-cyanoimino-1,3-thiazolidine. The wet crystals are dried in vacuo at 80° C. under reduced pressure for 5 hours to obtain 1.14 g of 2-cyanoimino-1,3-thiazolidine (yield 65%) with >99% HPLC purity.

### Example 10

A 1 litre four-necked flask provided with a thermometer and stirrer is charged with 100 g of methanol, and 41.6 g of sodium carbonate (0.393 moles, 1.15 mole equivalent based upon 1.09 mole equivalent of 2-aminoethane thiol hydrochloride). The mixture is cooled and stirred to dissolve in methanol, then 42 g of 2-aminoethane thiol hydrochloride (0.373 moles) was added thereto and dissolved therein, the reaction mixture is now cooled to 0° C. To this reaction mixture, 50 g of dimethyl N-cyanoiminodithiocarbonate (0.342 moles) is added keeping the temperature inside the system at 5° C. or less. After the end of addition, the mixture is allowed to react at 0 to 5° C. for 2 hours. Thereafter, the reaction mixture is heated to 20° C., the pH is adjusted to 4 with aqueous hydrochloric acid solution (36% w/w), the reaction mass then further heated to 40° C. and stirred for 2 hours. It is cooled to 0° C., under stirring the precipitated crystals are suction filtered and slurry washing with 150 ml of chill water to obtain 40 g of 2-cyanoimino-1,3-thiazolidine. The wet crystals are dried in vacuo at 80° C. under reduced pressure for 5 hours to obtain 32.5 g of 2-cyanoimino-1,3-thiazolidine (yield 75%) with 99.4% HPLC purity.

### Example 11

A 3 liter four-necked flask provided with a thermometer and a stirrer is charged with 238 g of dimethyl N-cyanoiminodithiocarbonate (1.62 moles) and 500 g of water (27.77 moles), and stirred at 0° to 10° C. To this reaction mixture, 184 g of 2-aminoethane thiol hydrochloride (1.62 moles) in equal quantity of water and 25% of aqueous ammonia (110.6 g) are charged in one lot. The reaction starts instantaneously with evolution of methyl mercaptan, which is trapped in equimolar aqueous caustic lye, or methanolic solution of caustic trap. The reaction is carried out preferably at 5° C. and continued at ambient temperature for 2 hours. The reaction mixture cooled to room temperature, followed by nitrogen purging for complete evacuation of methyl mercaptan for 2 hours. The solid compound is precipitated as faint grayish in colour, which is recrystallised from hot water. The solid is filtered and dried at 80° C. for 6-8 hours till moisture is below 0.5% by K.F. to obtain 170.76 g of 2-cyanoimino-1,3-thiazolidine (yield 83%) and having 98 to 99% (w/w) HPLC purity.

### ADVANTAGE OF THE PRESENT INVENTION

The previously described versions of the subject matter and its equivalent thereof have many advantages, including those which are described below
a) The process of the present invention uses reduced volumes of solvent to achieve good yields of substantially pure 2-cyanoimino-1,3-thiazolidine.
b) The process of the present invention does not employ corrosive reagents like alkali metal hydroxides.
c) The methyl mercaptan formed as byproduct in the process is isolated as sodium salt which can be used in the preparation of agrochemicals.

What is claimed is:

1. A process for the preparation of 2-cyanoimino-1,3-thiazolidine said process comprising dissolving a base in solvent, adding 2-aminoethanethiol of formula II or salts thereof 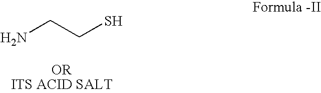
and dimethyl N cyanoiminodithiocarbonate ester of formula III 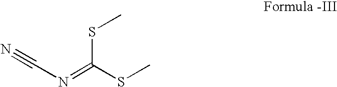
in an inert atmosphere to obtain a reaction mixture, adjusting pH of the reaction mixture with a mineral acid, stirring and heating to obtain substantially pure 2-cyanoimino-1,3-thiazolidine, wherein the molar ratio of base to 2-aminoethanethiol or salts thereof is from 1:1 to 1:1.5 and the molar ratio of dimethyl N cyanoiminothiocarbonate to 2-aminoethanethiol or salts thereof is from 1:0.95 to 1:1.15.

2. The process as claimed in claim 1 wherein the base is an alkali metal alkoxide selected from sodium methoxide, sodium ethoxide, potassium methoxide, potassium ethoxide, sodium butoxide, sodium isobutoxide or potassium butoxide.

3. The process as claimed in claim 1 wherein the base is an alkali or alkaline earth metal carbonate selected from sodium carbonate, potassium carbonate, magnesium carbonate, calcium carbonate or ammonium carbonate.

4. The process as claimed in claim 1 wherein the solvent is selected from C₁-C₄ straight or branched chain alcohol, tetrahydrofuran, toluene, water or a mixture thereof.

5. The process as claimed in claim 1 wherein the salt of the compound of formula II is selected from hydrochloride, sulfate, nitrate, carbonate or acetate.

6. The process as claimed in claim 1 wherein the mineral acid is selected from hydrochloric acid, sulfuric acid, nitric acid, carbonic acid or phosphoric acid.

7. The process as claimed in claim 1 wherein the pH of the reaction mixture is 3-10.

8. A process for the preparation of 2-cyanoimino-1,3-thiazolidine, said process comprising dissolving dimethyl N-cyanoiminodithiocarbonate ester of formula II in a solvent, 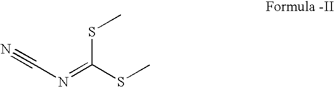
adding 2-aminoethanethiol or salts thereof of formula III 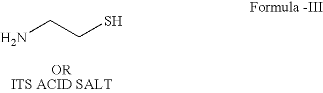
to a mixture of water and aqueous ammonia, reacting the solution of ester of formula II and solution of formula III at 0-35° C. for 2 hours to obtain 2-cyanoimino-1,3-thiazolidine, wherein the molar ratio of dimethyl N-cyanoiminodithiocarbonate ester to 2-aminoethanethiol acid salt is from 1:0.95 to 1:1.15.

9. The process as claimed in claim 8 in which 2-cyanoimino-1,3-thiazolidine is subjected to recrystallised from hot water to obtain substantially pure form of 2-cyanoimino-1,3-thiazolidine.

10. The process as claimed in claim 8, wherein the molar ratio of dimethyl N-cyanoiminodithiocarbonate ester to 2-aminoethanethiol acid salt is 1:1.

11. The process as claimed in claim 8 wherein the aqueous ammonia is 18-25% w/w.

12. The process as claimed in claim 8 wherein the acid salt is a hydrochloride salt.

13. The process as claimed in claim 8 wherein the solvent is water.

14. The process as claimed in claim 8 wherein the quantity of aqueous ammonia and water in the said mixture is equal.

15. The process as claimed in claim 1 or 8 wherein time of reaction is 10 min to 5 hrs.
